(19) United States
(12) Patent Application Publication
(10) Pub. No.: US 2016/0143590 A1
(43) Pub. Date: May 26, 2016

(54) ELECTROCARDIOGRAPHY SYSTEM

(71) Applicant: BIOTRONIK SE & CO. KG, Berlin (DE)

(72) Inventors: Jens PHILIPP, Berlin (DE); Stephan FANDREY, Affoltern am Albis (CH); Marc BARTELS, Berlin (DE)

(73) Assignee: BIOTRONIK SE & CO. KG, Berlin (DE)

(21) Appl. No.: 14/946,649

(22) Filed: Nov. 19, 2015

Related U.S. Application Data
(60) Provisional application No. 62/084,582, filed on Nov. 26, 2014.

(51) Int. Cl. A61B 5/00; A61B 5/044; A61B 5/055; A61B 8/06; A61B 5/042; A61B 5/024; A61B 5/026; A61B 5/04; A61B 7/02

(52) U.S. Cl. CPC A61B 5/7203; A61B 5/04012; A61B 5/044; A61B 5/055; A61B 7/02; A61B 5/042; A61B 5/02416; A61B 5/026; A61B 8/06

(57) ABSTRACT

Embodiments include an electrocardiography system that includes or is connected to a sensor arrangement, wherein the sensor arrangement includes surface electrodes that record electric signals. The electrocardiography system includes a signal processing unit, with filters and amplifiers, which is electrically connected to the sensor arrangement. The electrocardiography system includes a signal evaluation unit connected via a first signal input to the signal processing unit. The signal evaluation unit processes one or more prepared ECG signals provided by the signal processing unit in order to recover an electrocardiogram signal and to display the electrocardiogram signal. The signal evaluation unit includes a second signal input that receives secondary signals obtained by body sensors or monitors, wherein the signal evaluation unit processes the prepared ECG signals depending on the secondary signals.

This application claims the benefit of U.S. Provisional Patent Application 62/084,582 filed on 26 Nov. 2014, the specification of which is hereby incorporated herein by reference.

### BACKGROUND OF THE INVENTION

1. Field of the Invention

Embodiments of the invention generally relate to an electrocardiography system with a sensor arrangement that includes surface electrodes to record electric signals.

2. Description of the Related Art

Generally, known electrocardiography systems are used to record cardiograms. To this end, typically, surface electrodes are applied to the skin of a patient and electric potentials are detected, which are recorded as electric signals and are prepared for example by amplification and filtering. Generally, the prepared signals are then further processed and for example are presented to a doctor in the form of an electrocardiogram.

Typically, the electrocardiogram (ECG) shows the electric potential changes occurring with the contraction and expansion of heart chambers and atriums and therefore provides enlightening information concerning the respective acute state of the heart. It is therefore, generally, usual to record electrocardiograms continuously, even during other examinations or treatments. Typically, besides fluoroscopy, the n-channel ECG (n>1) is therefore the most important instrument in an electrophysiological examination (EPE) in order to diagnose cardiological arrhythmias, for example with catheterization and ablation.

Generally, magnetic resonance tomography, also known as MRT, is an image producing method of the magnetic resonance tomography as well as the therefore necessary magnetic resonance tomographs, wherein volume images even of soft tissue may be recorded in real time. To this end, typically, the object to be examined or the person to be examined is exposed in a magnetic resonance imaging (MRI) scanner to strong, changing magnetic fields. Generally, magnetic resonance imaging (MRI) provides a spatially and temporally resolved soft tissue contrast and thus enables a broad spectrum of diagnostic applications for organs or anatomies that are difficult to access, such as the brain, the spinal column, the joints, the abdomen, the cardiovascular system, etc.

Typically, the recording of electrocardiograms during the execution of magnetic resonance imaging in an MRI scanner, which is desirable per se, is not so easy however, because, during the measurement of electrocardiogram (ECG) signals in the MRI scanner, signal interference may occur, caused inter alia by the magneto hydrodynamic effect (MHD effect). Generally, this effect is produced by the high static primary magnetic field in the magnetic resonance imaging (MRI) scanner.

Typically, a combination of both medical measurement methods would make it possible to perform an EPS and MRI up to interventional or intraoperative MR (iMR) with the objective of performing ablations more precisely and particularly in a manner that is non-ionizing for the patient and hospital staff.

Generally, the direct combination of ECG and MRI poses technical challenges for physical reasons alone, in particular due to the fact that the three main components of the MRI scanner are independent of one another and consequently may be addressed separately. For example, typically, the three main components include the strong homogenous static magnetic field in the Tesla range (1), the three magnetic gradient fields directed orthogonally to one another (2), and the electromagnetic high-frequency fields (3).

Generally, the magneto hydrodynamic effect (MHD effect) is caused physically by moved charge carriers in a magnetic field that are forced by the Lorentz force in a certain direction, depending on the vectorial direction of movement of the charge carriers and the orientation of the magnetic field. Typically, the MHD effect, as a pseudo-periodic electrophysiological disturbance variable in the ECG, is induced primarily by the blood flow in the aortic arch during systole. Generally, moved charge carriers in the blood are pressed against the blood vessel wall by the Lorentz force and temporarily accumulate there locally. Typically, this temporary charge density superimposes, as potential, the ECG to be recorded, in particular in the QRS complex thereof, and may be measured with the ECG on the skin surface of the patient.

Generally, a hardware solution for eliminating the MHD contribution from the ECG uses integrated 3-channel ECG monitors, which are based on optical fibers and which are suitable exclusively for ensuring a monitoring of the heart rate of sedated patients or patients at risk or for enabling synchronized heartbeat-based imaging control (gating). Typically, key morphological analyses for EPSs are not possible due to the poor signal quality.

Generally, a 3-channel monitor is also known that indeed may also be operated in the examination room of the MRI scanner, but cannot be used above a field strength of 40 mT and therefore in the isocenter of the magnet in the MRI scanner.

Typically, two principle solution approaches for filtering the MHD effect have been suggested, including mathematical model-based approaches and conventional digital filters. Generally, mathematical approaches start by modeling the blood flow in the aorta and then predict from this the MHD effect in order to remove it from the distorted ECG. For example, “Realistic MHD Modeling Based on MRI Blood Flow Measurements”, a publication of The International Society for Magnetic Resonance in Medicine 20th Scientific Meeting & Exhibition, 2012 May, Melbourne, Australia, based on a human torso model, to Oster et al., presents research with an improved mathematical model, of an already provided mathematical model, of the MHD effect and of the ECG with an additional aorta model, and has validated this with real MRI data supported by contrast agent. According to Oster et al., the blood flow was determined in the MRI data with two regions of interest (ROIs) both in the ascending and in the descending aorta and was transferred to the aorta as a whole. As described in Oster et al., the potentials of the MHD effect and of the ECG were then calculated from the quantified blood flow.

Generally, various non-patent literature regarding digital filters have set about training an (adaptive) filter. For example, the non-patent literature include Adaptive Least Mean Square (LMS) Filtering, see for example “A 1.5T MRI-Conditional 12-Lead Electrocardiogram for MRI and Intra-MR Intervention”, a publication of Magnetic Resonance in Medicine, Volume 71, Issue 3, pages 1336-1347, March 2014, Adaptive Noise Cancellation (ANC), see for example “Adaptive noise cancellation to suppress electrocardiography artifacts during real-time interventional MRI”, a publication of the Journal of Magnetic Resonance Imaging, 2011 May, 33(5), pages 1184-1193, Wiener Filtering, see for example “The limited applicability of Wiener filtering to ECG signals disturbed by the MHD effect”, a publication of the Proc. of EUSIPCO 2012 Conference, August 27-31, Bucharest, Romania, and Template Matching, see for example “Extraction of the magnetohydrodynamic blood flow potential from the surface electrocardiogram in magnetic resonance imaging”, a publication of Medical & Biological Engineering & Computing, July 2008, Volume 46, Issue 7, pages 729-733.

Typically, if the objective is to separate cleanly from one another the ECG signals and disturbance signals induced by the MHD effect and to utilize this for diagnostic purposes, the known solutions from the industry are insufficient. Generally, the 3-channel ECGs, which may be used as monitors for patient monitoring and for synchronized imaging of MRI scanners (gating), are provided with the explicit instruction from the manufacturer not to derive any cardiological diagnostic characteristic variables, key for EPSs, or morphological analyses of the heart from the measurements.

Typically, the mathematical approaches in science have contributed significantly to the theoretical understanding of the influence of external and internal (bio)physical variables with regard to the MHD effect. Generally, the described models, however, show the complexity of the hemodynamic sequences of successive heartbeats of an individual only insufficiently, since unpredictable events, such as spontaneous arrhythmias or arrhythmias induced in a controlled manner during an EPS, for example by selective stimulation of the heart, the individual anatomy of the patient, and also extrasystoles, etc., could prevent a correct real-time separation of ECG and MHD effect.

Typically, filter-based solutions from the research are next in line from the practical viewpoint of a separation of ECG and MHD effect and adapt successful approaches from other technical fields, such as telecommunications or data compression. Generally, the pure transfer of the technology has proven to be inadequate, since it is unable to cope with the depolarization of the individual heart cells.

In addition, typically, all solution approaches in the case of the separation of MHD effect and ECG have the common feature that neither one nor more additional direct or indirect independent measured variables corresponding to the blood flow are included, fed back in real time, in a filter.

### BRIEF SUMMARY OF THE INVENTION

One or more embodiments of the invention provide a system that measures a diagnostic ECG in an MRI scanner, and therefrom, for example, an electrophysiological examination (EPE).

At least one embodiment of the invention includes an electrocardiography system with a sensor arrangement. In one or more embodiments, the sensor arrangement may include surface electrodes that record electric signals. In at least one embodiment, the electrocardiography system may be connected to the sensor arrangement. In one or more embodiments, the electrocardiography system may include a signal processing unit, for example with filters and amplifiers, wherein the signal processing unit may be electrically connected to the sensor arrangement. In at least one embodiment, the electrocardiography system may include a signal evaluation unit, which may be connected via a first signal input to the signal processing unit. In one or more embodiments, the signal evaluation unit may process one or more prepared ECG signals provided by the signal processing unit in order to recover an electrocardiogram signal and one or more of display the electrocardiogram signal and output the electrocardiogram to be displayed. The signal evaluation unit, in at least one embodiment, may include a second signal input that receives secondary signals obtained using body sensors or monitors. In one or more embodiments, the signal evaluation unit may process the prepared ECG signals in accordance with the secondary signals.

In at least one embodiment, the signal evaluation unit may include, form or adapt a compensation function in accordance with the secondary signals, and may apply the compensation function to the prepared ECG signal.

By way of one or more embodiments, with the aid of the secondary signals recovered and provided by additional independent sensors, in real time, an ECG falsified in the MRI scanner may be corrected by determining and compensating for the influence of the magneto hydrodynamic effect (MHD effect). In at least one embodiment, for the ECG in the MRI scanner, a falsified measured signal, caused by MHD-induced disturbance signals, may be obtained. In one or more embodiments, the falsified measured signal may be represented as ECG(MRI)=ECG+MHD, expressed in the measured voltages as V_{ECG(MRI)}=V_{ECG}+V_{MHD}.

In at least one embodiment, the signal evaluation unit may include an adaptive filter, which may be adapted by coupling the ECG measurement with initial filter/transfer function using the secondary signals. In one or more embodiments, the adaptive filter may be provided effectively with relatively a few additional hardware outlay by use of the intracardial ECG already provided with the electrophysiological examination (EPE) and the additional sensors.

By way of at least one embodiment, for a healthy, regularly beating heart, a transfer function between the measurement in the MRI scanner and outside may be established. In one or more embodiments, the transfer function may function as long as no irregularities occur, wherein the signal remains predictable. In at least one embodiment, during an electrophysiological (EP) procedure, the signal may not be predictable, since arrhythmias may occur, for example by the procedure itself, such as by selective stimulation of the heart, and also because the heart is almost always a pathologically modified heart.

As such, one or more embodiments of the invention may perform an initial filter calculation by detecting the change of the ECG measurement at the patient when introduced into the MRI scanner. In at least one embodiment, the initial filter calculation may then be adapted continuously by independent sensors and measurements. In one or more embodiments, the independent sensors may deliver the secondary signals, and may not be influenced by the MHD effect. At least one embodiment may include one or more independent sensors in order to train the adaptive filter function and to improve the ECG measurement.

By way of one or more embodiments, the electrocardiography system may perform a parallel measurement and correction of an n-channel electrocardiogram (ECG n>1) in a magnetic resonance imaging (MRI) scanner for diagnostic purposes and simultaneous use of the magneto hydrodynamic effect (MHD effect) for hemodynamic diagnostic statements. In at least one embodiment, the parallel measurement and correction may include simultaneous scanning of the anatomy of the patient by the MRI scanner, an automatic segmentation of the aorta and calculation of the MHD effect from the blood flow under consideration of further physiological real-time measurements with imaging methods independent of the ECG.

One or more embodiments of the invention provide disturbance-free measurement of the electrophysiological signals in the environment of strong homogenous static magnetic fields, which may be falsified by the MHD effect. In at least one embodiment, the signal evaluation unit may clean up an n-channel electrocardiogram (ECG, n>1) recorded in the environment of strong magnetic fields. In one or more embodiments, the n-channel electrocardiogram may have been distorted by induced magneto hydrodynamic disturbance signals. In at least one embodiment, the signal evaluation unit may clean up the n-channel electrocardiogram to draw diagnostic conclusions concerning both the hemodynamics and the electrophysiology of the heart in an environment of strong static magnetic fields.

In one or more embodiments, the sensor arrangement may include secondary sensors, which are different from the surface electrodes, to detect a further physical variable. In at least one embodiment, the secondary sensors may deliver one or more secondary signals. In one or more embodiments, the signal evaluation unit may include an adaptive filter, of which the transfer function is influenced and/or controlled by the secondary signal or the secondary signals. In at least one embodiment, the adaptive filter may allow a measurement of a surface-ECG signal without MHD-induced disturbance signals. In one or more embodiments, the adaptation of the filter may be achieved by an evaluation of one or more independent sensors.

By way of at least one embodiment, the adaptive filter may include a transfer function that compensates for a transfer function between ECG signals recorded outside an MRI scanner and ECG signals recorded within the magnetic fields. In one or more embodiments, the transfer function of the adaptive filter may be an inverse to the transfer function caused by the influence of the MRI scanner on the ECG, such as the ECG signals recorded in the MRI scanner.

In at least one embodiment, the signal evaluation unit may form an initial transfer function of the adaptive filter by detecting the change of the ECG measurement at patients when introduced into the MRI scanner.

In one or more embodiments, the secondary sensor may record an intracardial ECG and may deliver an intracardial ECG as a secondary signal. In at least one embodiment, an intracardial ECG may deliver values suitable for the adaptive filter calculation. In one or more embodiments, an intracardial ECG may be recorded via additional diagnostic catheters as secondary sensors, which may be used during an EP procedure. According to at least one embodiment, due to the small vector between the electrodes with the signal recording of the intracardial ECG, the MHD effect may have no influence in the MRI scanner during the measurement. In one or more embodiments, since the surface-ECG and the intracardial ECG are associated with one another, the joint ECG may be used as a measured value independent of the MHD effect during adaptive filter calculation. In at least one embodiment, changes in the cardiac rhythm may be identified in the intracardial ECG and therefore may be used for the calculation.

In one or more embodiments, the secondary sensor may include or may be an acoustic sensor, for example to record heart sounds. In at least one embodiment, the heart sounds are not influenced by the MHD effect and therefore may be used for filter calculation and adaptation.

By way of one or more embodiments, the secondary sensor may include or may be a pulse oximeter, for example to measure a pulse. In at least one embodiment, the pulse is not influenced by the MHD effect and therefore may be used for filter calculation and adaptation.

In at least one embodiment, acoustic sensors and pulse oximeters are examples of secondary sensors that may be used as additional sensors independent of the MHD effect, which may deliver the secondary signals that may be included into the mathematical model from the adaptive filter.

In at least one embodiment, the second signal input may be connected to a measurement device that measures real-time blood flow, wherein the secondary signals may represent a blood flow in real time. In one or more embodiments, the signal evaluation unit may determine at least one compensation signal from the secondary signals which reflects the influence of the magneto hydrodynamic effect (MHD), and may subtract the compensation signal from one or more prepared ECG signals or may otherwise recalculate the signals in a compensatory manner.

As such, in at least one embodiment of the invention, ECGs and disturbance signals induced by the MHD effect in the environment of strong magnetic fields may be separated to then utilize both electrophysiological signals for diagnostic purposes. In one or more embodiments, the blood flow corresponding to the MHD effect may be detected via independent medical imaging in real time, for example in the MRI scanner, may be quantified by the signal evaluation unit and may be supplied as a fed-back variable to a filter or a filter cascade.

In at least one embodiment, the measurement device that measures real-time blood flow may be, for example, an MRI scanner, which delivers digital imaging and communications in medicine (DICOM) data. In one or more embodiments, the DICOM data may include the secondary signal, as discussed herein.

According to at least one embodiment of the invention, the measurement device that measures real-time blood flow may be an ultrasound measuring device, which performs an independent non-ionizing ultrasound measurement.

In one or more embodiments, the signal evaluation unit may evaluate DICOM data originating from the MRI scanner as the secondary signal, for example wherein the signal evaluation unit may automatically segment the aorta and calculate the MHD from the blood flow in the aorta.

By way of at least one embodiment of the invention, the electrocardiography system may allow the parallel measurement and correction of an n-channel electrocardiogram (ECG, n>1) in a magnetic resonance imaging (MRI) scanner for diagnostic purposes, and the simultaneous use of the magneto hydrodynamic effect (MHD) for hemodynamic diagnostic statements. For example, in one or more embodiments, the electrocardiography system may scan the anatomy of the patient by the MRI scanner, may automatically segment the aorta, and may calculate the MHD effect from the blood flow under the consideration of further physiological real-time measurements with imaging methods independent of the ECG.

One or more embodiments, as an advantage of the electrocardiography system discussed herein, may include an addition of one or more independent measured variables corresponding to the blood flow. In at least one embodiment, the one or more independent measured variables may be additionally supplied, fed back in real time, to a filter or a filter cascade in order to separate the n-channel ECG (n>1) from the disturbance signals induced by the MHD effect, in order to draw diagnostic conclusions. As another advantage, embodiments of the invention may include integration of independent imaging methods. Generally, using known devices, it is theoretically impossible to calculate the MHD effect from the falsified ECG based on the non-stationary related sources thereof, and this may be implemented in practice only as an approximation.

### BRIEF DESCRIPTION OF THE DRAWINGS

The above and other aspects, features and advantages of at least one embodiment of the invention will be more apparent from the following more particular description thereof, presented in conjunction with the following drawings, wherein:

FIG. 1 shows an overview of an electrocardiography system according to one or more embodiments of the invention;

FIG. 2 shows a diagram that explains the operating principle of a signal evaluation unit according to one or more embodiments of the invention;

FIG. 3 shows a first variant of an electrocardiography system according to one or more embodiments of the invention; and,

FIG. 4 shows a second variant of an electrocardiography system according to one or more embodiments of the invention.

### DETAILED DESCRIPTION OF THE INVENTION

The following description is of the best mode presently contemplated for carrying out at least one embodiment of the invention. This description is not to be taken in a limiting sense, but is made merely for the purpose of describing the general principles of the invention. The scope of the invention should be determined with reference to the claims.

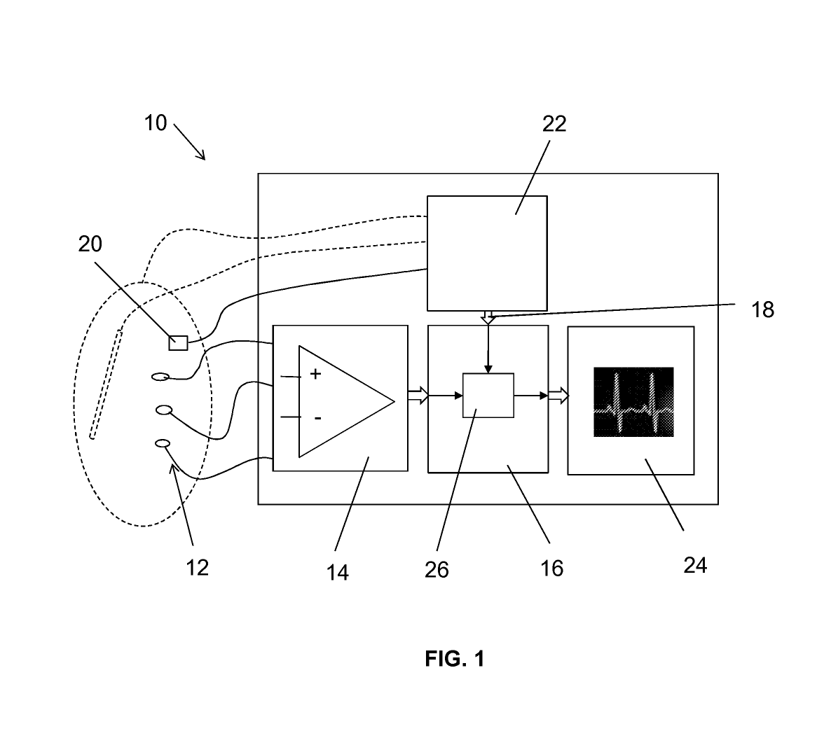

FIG. 1 shows an overview of an electrocardiography system 10 according to one or more embodiments of the invention. As shown in FIG. 1, in at least one embodiment of the invention, the electrocardiography system may include a sensor arrangement 12 of surface sensors that record electric signals, a signal processing unit 14 and a signal evaluation unit 16. In one or more embodiments, the sensor arrangement may be connected to the signal processing unit 14. In at least one embodiment, the signal processing unit 14 may amplify and filter the recorded electric signals. In one or more embodiments, the electric signals that are processed may be fed to the signal evaluation unit 16. In at least one embodiment, the signal evaluation unit may evaluate the recorded and processed electric signals under consideration of secondary signals, indicated by the arrow 18.

By way of one or more embodiments, the secondary signals 18 may originate from a secondary sensor 20 or from a plurality of different secondary sensors. At least one embodiment of the invention may include a secondary signal processing unit 22 that processes the signals delivered from the secondary sensor 20 and delivers the corresponding secondary signals 18 to the signal evaluation unit 16.

In one or more embodiments, the signal evaluation unit 16 may determine, from the processed delivered signals of the sensor arrangement 12 and the secondary signals 18, an electrocardiogram (ECG), partially or completely, cleaned up of influence of a magneto hydrodynamic effect (MHD). In at least one embodiment, the electrocardiogram (ECG) may one or more of be displayed on a display and transmitted to a printer 24.

In at least one embodiment of the invention, the signal evaluation unit 16 may include an adaptive filter 26. In one or more embodiments, the adaptive filter 26 may be adapted in a controlled manner or may at least be influenced by the secondary signals 18.

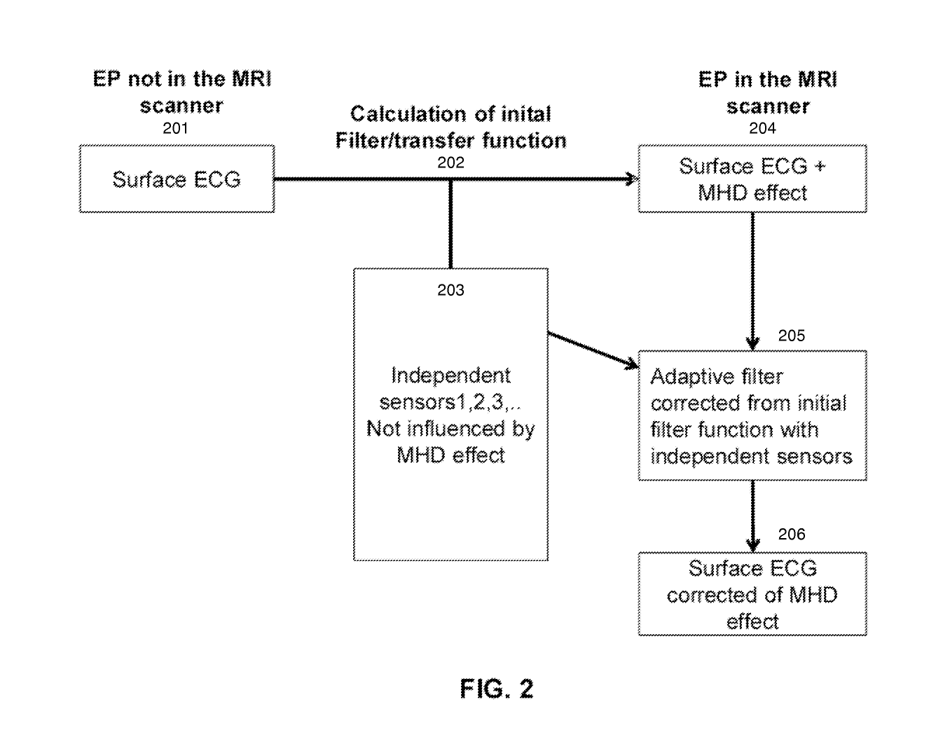

FIG. 2 shows a diagram that explains the operating principle of a signal evaluation unit according to one or more embodiments of the invention. According to at least one embodiment, the adaptive filter 26 may include an initial transfer function, which the signal evaluation unit 16 may have determined from a change of the delivered electric signals upon introduction of a patient into a magnetic resonance imaging (MRI) scanner. Prior to the introduction of the patient into the MRI scanner, at 201, in one or more embodiments, the delivered electric signals may not yet be influenced by the MRI scanner and in particular by the MHD effect, at 203, whereas, after introduction of the patient into the MRI scanner, at 204, the delivered electric signals may be influenced by the MHD effect. At least one embodiment of the invention may compare the electric signals delivered prior to the introduction of the patient into the MRI scanner, at 201, with the electric signals delivered in the MRI scanner, at 204, in order to determine the influence of the MHD effect and to derive therefrom a transfer function. In one or more embodiments, the transfer function describes the changes of the delivered electric signals prior to and following introduction into the MRI scanner, at 202. At least one embodiment may include an inverse transfer function that may compensate for the influence of the MHD effect and therefrom may form an initial transfer function of the adaptive filter 26.

By way of one or more embodiments, at 205, the initial transfer function of the adaptive filter 26 may be corrected continuously and in real time, for example during an electrophysiological examination (EPE) of a patient in the MRI scanner, with the aid of at least one of the secondary signals 18, as shown in FIG. 2. By way of one or more embodiments, with the aid of the secondary signals recovered and provided by additional independent sensors at 203, in real time, an ECG falsified in the MRI scanner may be corrected by determining and compensating for the influence of the magneto hydrodynamic effect (MHD effect) at 205 and 206.

In at least one embodiment, a suitable secondary signal may be, for example, an intracardial electrocardiogram, which, by contrast to a surface electrocardiogram, is hardly influenced by the magneto hydrodynamic effect (MHD effect) due to the small vector between the intracardial electrode during the signal recording. In one or more embodiments, since the surface electrocardiogram and the intracardial electrocardiogram are associated, the intracardial electrocardiogram may be used as a secondary signal, which is largely independent of the magneto hydrodynamic effect, for adaptive filter calculation.

In at least one embodiment, if the secondary signal is an intracardial electrocardiogram, the secondary sensor 20 may be, for example, an additional diagnostic catheter, which may be used in electrophysiology procedures.

According to one or more embodiments, the secondary sensor may be, or may include, an acoustic sensor that records heart sounds or a pulse oximeter that measures the pulse. In at least one embodiment, both an acoustic sensor and a pulse oximeter may be suitable secondary sensors, because both the acoustic sensor and the pulse oximeter may deliver secondary signals independent of the MHD effect.

One or more embodiments may include a plurality of secondary sensors 20, as indicated by the dashed illustration in FIG. 1. In at least one embodiment, the secondary sensors 20 may include a combination of a probe that records an intracardial electrocardiogram and an acoustic sensor.

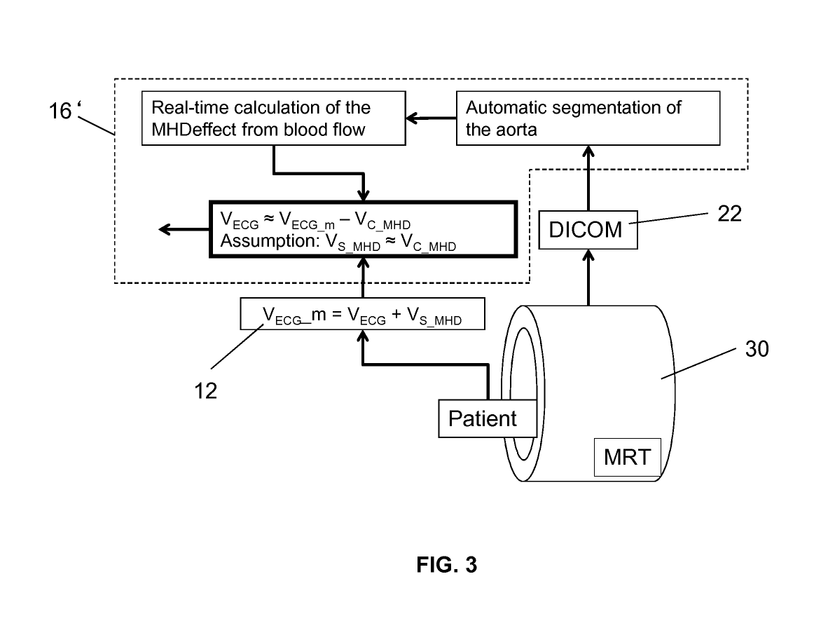

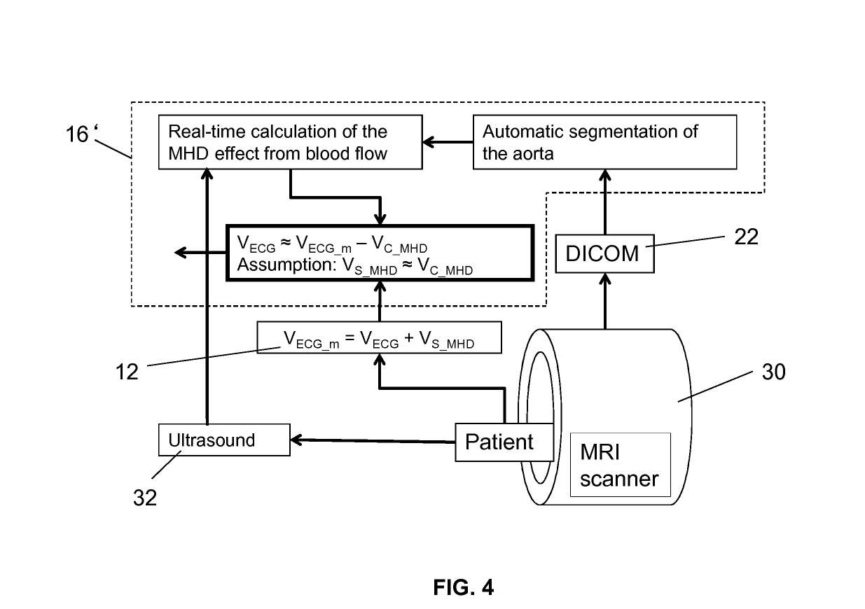

FIGS. 3 and 4 illustrate variants of an electrocardiography system according to one or more embodiments of the invention. As shown in FIGS. 3 and 4, in at least one embodiment, the secondary signal may be obtained via the magnetic resonance imaging (MRI) scanner itself to determine the current blood flow in the aorta from the DICOM data 22 delivered from the MRI scanner. In one or more embodiments, the signal evaluation unit 16′ may automatically segment the aorta from the DICOM data and may determine the blood flow in the aorta in real time. In at least one embodiment, via the signal evaluation unit 16′, the magneto hydrodynamic effect (MHD effect) may be determined from the blood flow in the aorta. As such, one or more embodiments, for example via the signal evaluation unit 16′, may determine the influence of the magneto hydrodynamic effect on the delivered and processed signals and may remove the influence from the delivered and processed electric signals. As such, in at least one embodiment, an electrocardiogram largely corrected of the magneto hydrodynamic effect is produced. FIG. 3, by way of one or more embodiments, shows a magnetic resonance imaging (MRI) scanner 30 as a secondary sensor.

In at least one embodiment, proceeding from a patient located in a MRI scanner, such as scanner 30, to which the surface electrodes that record an n-channel ECG (n>1) have already been attached, a four-dimensional (4D) MRI image sequence (for example phase contrast) may be recorded from the thorax of the patient. One or more embodiments may include administration of a contrast agent. In at least one embodiment, the patient's aorta may be automatically identified from the resultant DICOM data 22 as the basis for the real-time blood flow measurement and may be segmented. In one or more embodiments, as shown in FIGS. 3 and 4, the ECG (ECG_m or V_{ECG}_{_}ₘ) that may be measured in the MRI scanner is the ECG of the patient (ECG or V_{ECG}) falsified additively by the induced disturbance signals of the MHD effect (S_MHD or V_{S}_{_}_{MHD}). As shown in FIGS. 3 and 4, in at least one embodiment, the disturbance signal of the MHD effect calculated by the real-time blood flow measurement (C_MHD or V_{C}_{_}_{MHD}) may be removed from the falsified measured ECG (ECG_m or V_{ECG}_{_}ₘ), under the assumption that S_MHD or, expressed as potential, V_{S}_{_}_{MHD} and C_MHD or, expressed as potential V_{C}_{_}_{MHD} are similar. In one or more embodiments, the falsified measured signal may be represented as ECG(MRI)=ECG+MHD, 12, expressed in the measured voltages as V_{ECG(MRI)}=V_{ECG}+V_{MHD}.

By way of at least one embodiment, the variant shown in FIG. 4 differs from the variant shown in FIG. 3 in that an ultrasound device 32 may be included as a further secondary sensor, as shown in FIG. 4. In one or more embodiments, the ultrasound device 32 may determine the blood flow by ultrasound measurement. In at least one embodiment, the determination of the blood flow by ultrasound may not be influenced by the MRI scanner and therefore may provide a further measured value as a secondary signal to calculate the influence of the MHD effect by the signal evaluation unit 16, 16′. Accordingly, in one or more embodiments, the influence of the MHD effect may be determined even more precisely with an electrocardiography system shown in FIG. 4 than with the electrocardiography system shown in FIG. 3.

At least one embodiment of the invention may include an adapted filter to compensate for the magneto hydrodynamic effect or may include determining the influence of the magneto hydrodynamic effect with the aid of real-time blood flow measurements in the MRI scanner, in order to find an electrocardiogram that is influenced minimally by the magneto hydrodynamic effect. One or more embodiments of the invention may include both an adapted filter to compensate the magneto hydrodynamic effect and determining the influence of the magneto hydrodynamic effect with the aid of real-time blood flow measurements in the MRI scanner.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

### LIST OF REFERENCE SIGNS

10 electrocardiography system
12 sensor arrangement
14 signal processing unit
16, 16′ signal evaluation unit
18 secondary signal
20 secondary sensor
22 secondary signal processing unit
24 display/printer
26 adaptive filter
30 magnetic resonance imaging (MRI) scanner
32 ultrasound device

What is claimed is:

1. An electrocardiography system comprising:
a sensor arrangement, wherein the sensor arrangement comprises surface electrodes that record electric signals;
a signal processing unit electrically connected to the sensor arrangement; and,
a signal evaluation unit connected via a first signal input to the signal processing unit;
wherein the signal evaluation unit is configured to
process one or more prepared electrocardiograph (ECG) signals provided by the signal processing unit in order to
obtain an electrocardiogram signal, and
display the electrocardiogram signal or output the electrocardiogram signal to be displayed,
wherein the signal evaluation unit comprises a second signal input that receives one or more secondary signals obtained via at least one secondary sensor, wherein the at least one secondary sensor comprise one or more body sensors or monitors and,
wherein the signal evaluation unit is further configured to process the prepared ECG signals in accordance with the secondary signals.

2. The electrocardiography system according to claim 1, wherein the signal evaluation unit is further configured to form or to adapt a compensation function in accordance with the secondary signals and to apply the compensation function to the prepared ECG signal.

3. The electrocardiography system according to claim 1, wherein
the sensor arrangement further comprises the at least one secondary sensor, wherein the at least one secondary sensor differs from the surface electrodes, wherein the at least one secondary sensor is configured to detect a physical variable and delivery the one or more secondary signals, and
the signal evaluation unit further comprises an adaptive filter with a transfer function, wherein the transfer function is one or more of influenced by and controlled by the one or more secondary signals.

4. The electrocardiography system according to claim 3, wherein the transfer function is configured to compensate for a transfer function between ECG signals recorded outside an MRI scanner and ECG signals recorded within magnetic fields of the MRI scanner.

5. The electrocardiography system according to claim 3, wherein the signal evaluation unit is further configured to form an initial transfer function for the adaptive filter, wherein a change of an ECG measurement of a patient introduced in the MRI scanner is detected to form the initial transfer function.

6. The electrocardiography system according to claim 3, wherein the at least one secondary sensor is further configured to record an intracardial ECG, and wherein the one or more secondary signals comprise an intracardial ECG.

7. The electrocardiography system according to claim 3, wherein the at least one secondary sensor is an acoustic sensor configured to record heart sounds.

8. The electrocardiography system according to claim 3, wherein the at least one secondary sensor is a pulse oximeter configured to measure a pulse.

9. The electrocardiography system according to claim 1, wherein the second signal input is connected to a measurement device configured to measure real-time blood flow, wherein the one or more secondary signals represent a blood flow in real time, and wherein the signal evaluation unit is further configured to
determine from the one or more secondary signals at least one compensation signal, wherein the at least one compensation signal reflects an influence of a magneto hydrodynamic effect (MHD effect), and
subtract the compensation signal from the prepared ECG signals that have been processed.

10. The electrocardiography system according to claim 9, wherein the measurement device configured to measure real-time blood flow is an MRI scanner.

11. The electrocardiography system according to claim 10, wherein the measurement device configured to measure real-time blood flow is an ultrasound measuring device.

12. The electrocardiography system according to claim 10, wherein the signal evaluation unit is further configured to evaluate data originating from the MRI scanner as the one or more secondary signals by automatically segmenting an aorta of a patient and calculating the MHD effect from blood flow in the aorta.
